(19) United States
(12) Patent Application Publication
(10) Pub. No.: US 2008/0200873 A1
(43) Pub. Date: Aug. 21, 2008

(54) Methods and Apparatus for Infusing the Interior of a Blood Vessel

(76) Inventors: Alejandro Espinosa, Miamia, FL (US); Peter K. Kratsch, Davie, FL (US); Eduardo A. Ampuero, Miami, FL (US); William M. Garcia de Quevedo, Miami, FL (US)

Correspondence Address: GORDON & JACOBSON, P.C., 60 LONG RIDGE ROAD, SUITE 407, STAMFORD, CT, 06902, US

(21) Appl. No.: 11/675,919

(22) Filed: Feb. 16, 2007

(51) Int. Cl. A61M 25/14

(52) U.S. Cl. 604/102.01

(57) ABSTRACT

An apparatus for treating blood vessels includes an elongate body having a proximal end, a distal end, and an infusion lumen extending there-through, a plurality of infusion holes in valved communication with the infusion lumen, and a valve member which is movable inside the infusion lumen from a first position in which some of the infusion holes are blocked to a second position where fewer of the infusion holes are blocked. According to one embodiment, the valve member is a tube coaxially disposed inside the infusion lumen. According to a second embodiment, the valve member includes a seal coupled to a wire. When the wire is pulled, more holes are unsealed. In both embodiments, an inflatable occlusion balloon is coupled to the distal end of the apparatus and an inflation lumen extends from it to the proximal end of the device. A method of the invention includes determining the length of the blood vessel to be treated and adjusting the valve member to unblock a number of infusion holes corresponding to the length of the vessel to be treated.

### CROSS-REFERENCE TO RELATED APPLICATIONS

This application is related to co-owned, co-pending application Ser. No. 11/624,412, filed Jan. 18, 2007 [VRX-006], entitled “Method for Infusing the Interior of a Blood Vessel”, which is hereby incorporated herein by reference in its entirety.

### BACKGROUND OF THE INVENTION

1. Field of the Invention

The invention relates to the treatment and correction of venous insufficiency. More particularly the invention relates to a minimally invasive procedure using a catheter-based system to treat the interior of a blood vessel. The invention has particular application to varicose veins although it is not limited thereto.

2. State of the Art

The above incorporated application discloses methods and apparatus for infusing the interior of a blood vessel. The methods are practiced with a catheter having an infusion lumen, a plurality of infusion holes, a movable barrier (preferably an inflatable bladder) between the infusion lumen and the infusion holes, a blood vessel occluder (preferably an occlusion balloon), and an inflation lumen for the blood vessel occluder. The methods include blocking the infusion holes with the inflatable barrier, inserting the catheter into the blood vessel, inflating the occlusion balloon, unsealing the infusion holes, injecting the therapeutic agent (preferably a sclerosant) through the infusion lumen, deflating the occlusion balloon and removing the catheter from the blood vessel. The methods also preferably include testing the occlusion balloon and priming the infusion lumen prior to sealing the infusion holes and inserting the catheter into the blood vessel. The balloon is preferably tested by purging air from the balloon, inflating it with saline or contrast media and inspecting it for leaks, then deflating it. The infusion lumen is preferably primed by unblocking the infusion holes and injecting the therapeutic agent until it flows through all of the infusion holes.

The step of inserting the catheter into the blood vessel is preferably preceded by inserting a sheath introducer and a guide wire into the blood vessel. The step of inserting also preferably includes locating the occlusion balloon under ultrasonic or fluoroscopic guidance before inflating it. Those skilled in the art will also appreciate that after the catheter is removed from the blood vessel and the sheath is also removed. The guide wire is removed prior to treatment.

According to the disclosure, all of the infusion holes are sealed and unsealed simultaneously. Those skilled in the art will appreciate that when treating different patients, the length of the vein which needs to be treated will vary. According to the previously preferred apparatus, catheters are supplied in nine different configurations where the number of infusion holes are as few as six or as many as twenty-two. In the case of six infusion holes, the “infusion length” is approximately 12 cm. In the case of twenty-two infusion holes, the infusion length is approximately 44 cm.

### SUMMARY OF THE INVENTION

It is therefore an object of the invention to provide a single treating apparatus which is adaptable to the different treatment needs of different patients.

It is another object of the invention to provide methods for adapting the treating apparatus to the different treatment needs of different patients.

It is a further object of the invention to provide a treating apparatus where the number of infusion holes which are unsealed is selectable by the treating practitioner.

It is still another object of the invention to provide a single length treating apparatus where the infusion length is selectable by the practitioner.

In accord with these objects, which will be discussed in detail below, an apparatus according to the present invention includes an elongate body having a proximal end, a distal end, and an infusion lumen extending there-through, a plurality of infusion holes in valved communication with the infusion lumen, and a valve member which is movable inside the infusion lumen from a first position in which some of the infusion holes are blocked to a second position where fewer of the infusion holes are blocked. According to one embodiment, the valve member is a tube coaxially disposed inside the infusion lumen. When the tube is moved proximally, more infusion holes are unblocked. When it is moved distally, more holes are blocked. According to a second embodiment, the valve member includes a seal coupled to a wire. When the wire is pulled proximally, more holes are unblocked.

In both embodiments, an inflatable occlusion balloon is coupled to the distal end of the apparatus and an inflation lumen extends from the proximal end of the device to the balloon. A guide wire lumen is also provided. The apparatus is preferably used in conjunction with an introducer sheath and a guide wire.

The methods of the invention include deploying an introducer sheath and a guide wire in the blood vessel to be treated and determining the length of the blood vessel to be treated. The valve member is adjusted to unblock a number of infusion holes corresponding to the length of the vessel to be treated and the device is primed. The apparatus is then inserted over the guide wire and through the introducer sheath into the blood vessel. The distal end of the apparatus is located by measurement, ultrasound, or fluoroscopy. The guide wire is then removed through the hub of the apparatus. The occlusion balloon is then inflated and a treating fluid is injected into the infusion lumen. After treatment, the occlusion balloon is deflated and the apparatus and the introducer sheath are removed from the blood vessel.

Additional objects and advantages of the invention will become apparent to those skilled in the art upon reference to the detailed description taken in conjunction with the provided figures.

### BRIEF DESCRIPTION OF THE DRAWINGS

FIG. 1 is a perspective view of a first embodiment of the apparatus of the invention;

FIG. 2 is an enlarged broken perspective, partially cut away and partially sectional, view of the insertable part of the apparatus of FIG. 1;

FIG. 3 is an enlarged broken perspective view of the proximal hub and sliding tube of the apparatus in FIG. 1;

FIG. 4 is an enlarged sectional view taken along line 4-4 in FIG. 1;

FIG. 5 is a perspective view of a second embodiment of the apparatus of the invention;

FIG. 6 is a broken plan view of the apparatus of FIG. 5;

FIG. 7 is an enlarged, broken, cut away and partially sectional, view of the distal end of the apparatus of FIG. 5;

FIG. 8 is an enlarged sectional view taken along line 8-8 in FIG. 6; and

FIG. 9 is an enlarged broken perspective. Partially cut away view of the distal end of the apparatus of FIG. 5.

### DETAILED DESCRIPTION OF THE PREFERRED EMBODIMENT

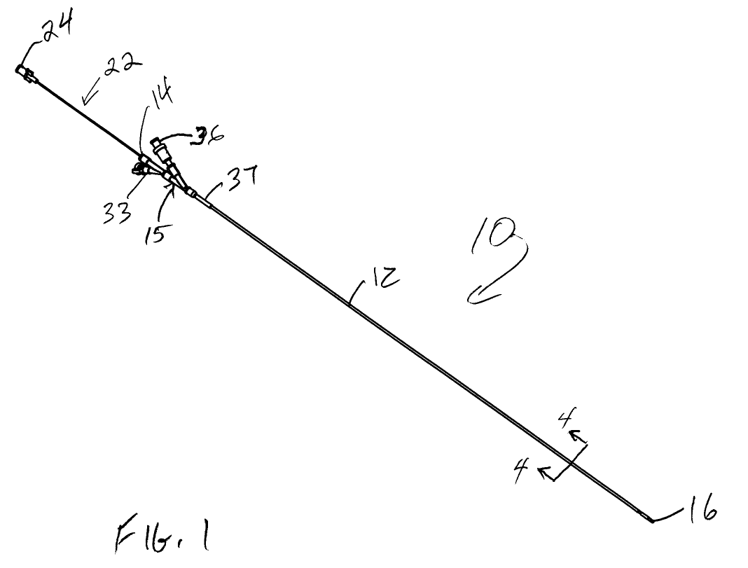

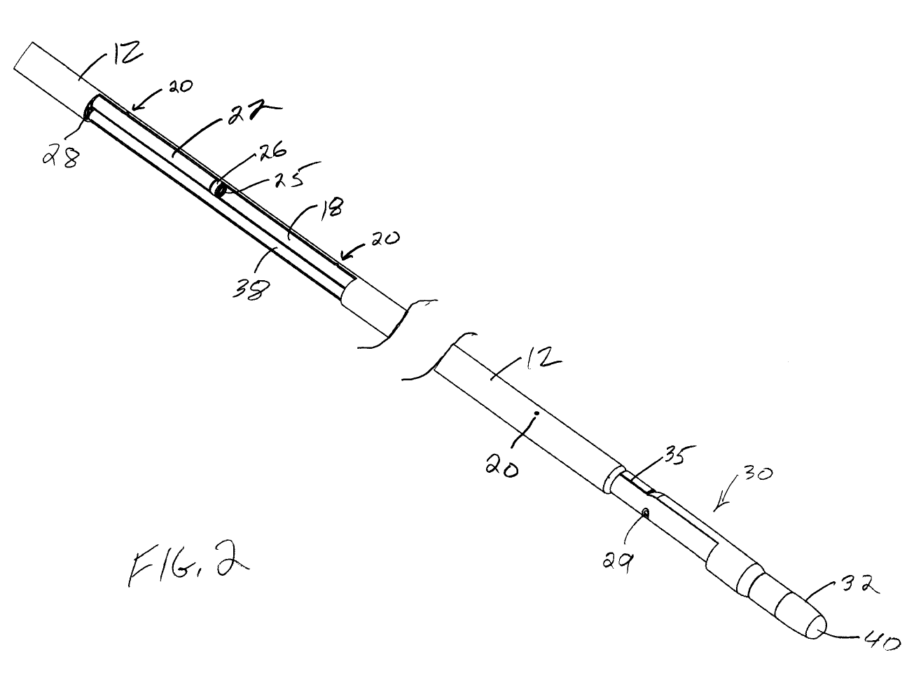

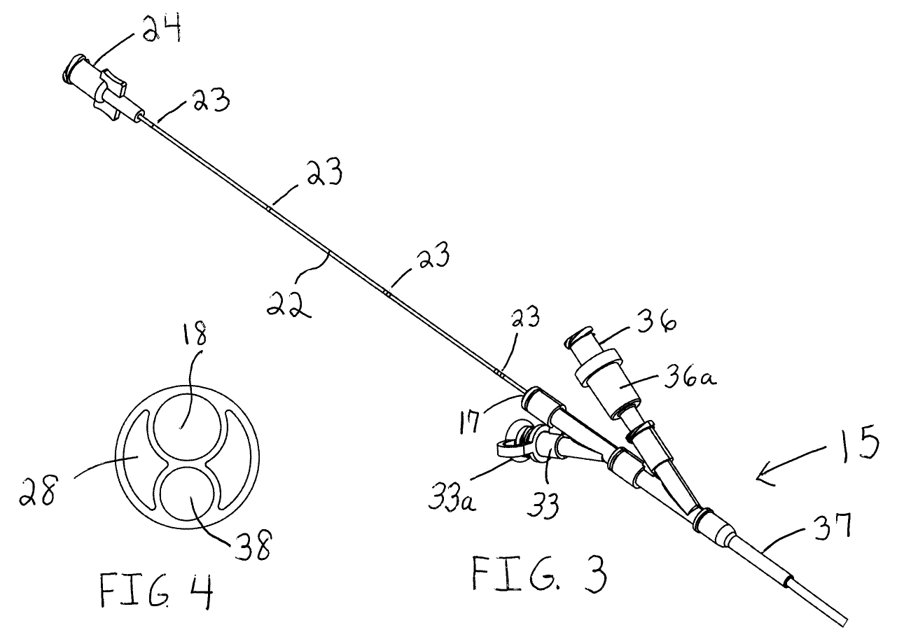

Turning now to FIGS. 1-4, an apparatus 10 for treating the interior of a blood vessel includes an elongate body 12 having a proximal end 14, a distal end 16, and an infusion lumen 18 extending there-through (FIGS. 2 and 4). A plurality of infusion holes 20 (FIG. 2) are in valved communication with the infusion lumen 18. A valve member 22 is movable inside said infusion lumen 18. In the first embodiment, the valve member 22 is a tube (non-perforate cylinder) which extends into the proximal end 17 (FIG. 3) of the infusion lumen 18 and is slidable through the infusion lumen 18. As seen best in FIG. 3, the proximal end of the tube 22 is provided with an infusion port 24 (a luer lock). As seen best in FIG. 2, the distal end of the tube 22 is provided with a fluid exit opening 25 which is surrounded by a sealing structure 26 which creates a fluid seal with the interior wall of the infusion lumen 18. The sealing structure 26 may be formed by flaring the distal end of the tube 22 or by providing it with an annular bulge or annular balloon or a rubber (or other compliant material) O-ring or similar annular seal. From the foregoing, those skilled in the art will appreciate that when a treating fluid is injected through the infusion port 24 it will flow through the tube 22, out the fluid exit opening 25 of the tube 22, into the infusion lumen 18 and out of those infusion holes 20 which are located distal of the sealing structure 26.

As seen best in FIGS. 1 and 3, the proximal end 14 of the elongate body 12 is provided with a hub 15. The hub is held by the practitioner in one hand while the tube 22 is moved into or out of the infusion lumen with the other hand to determine how many infusion holes will be used to infuse. To aid in positioning the tube relative to the infusion lumen, indicia 23 (e.g. stripes) are located along the tube 22. When the proximal end 17 of the infusion lumen is located on an indicium the number of infusion holes associated with the indicium are unblocked. Alternatively, the body 12 may be made transparent or translucent such that the location of the tube 22 can be readily ascertained. In such case, indicia on the body 12 may also be provided.

As seen best in FIGS. 2 and 4, the elongate body 12 is provided with a second lumen 28. The distal end of the second lumen 28 terminates inside an inflatable balloon 30 via a skive 29. An atraumatic tip 32 is coupled to the distal end of the balloon 30. The proximal end of the second lumen 28 is coupled to the side port 36 of the hub 15 shown in FIGS. 1 and 3. The side port 36 includes a luer with a luer activated valve 36a for connecting a balloon inflation syringe (not shown) and the end of the hub 15 is provided with a shrink tube 37 which acts as a strain relief between the hub 15 and the elongate member 12. The balloon 30 (FIG. 2) is annular and surrounds a guide wire lumen 38. The guide wire lumen 38 extends from a hole 40 in the tip 32 to the side port 33 of the hub 15. The side port 33 is provided with a plug cap 33a to close off the guide wire lumen when not in use.

From the foregoing, those skilled in the art will appreciate that the methods of using the apparatus 10 include the following steps. The blood vessel is measured and the infusion length is determined. The tube 22 is moved relative to the infusion lumen 18 to unblock a number of infusion holes corresponding to the infusion length. The occlusion balloon is tested and the infusion lumen is primed. A sheath introducer and guide wire are inserted into the blood vessel to be treated. Then, the guide wire is threaded through the hole 40 in the tip 32 and out through the side port 33 of the hub 15. The apparatus 10 is moved over the guide wire through the introducer sheath and into the blood vessel. The distal end of the device may be radiopaque to aid in locating it with fluoroscopy. Alternatively, it can be located by measurement or by ultrasound. The guide wire is then removed through the side port 33 of the hub 15 and the side port is plugged with the plug cap 33a. The occlusion balloon 30 is then inflated and a therapeutic fluid is infused through the infusion port 24. The balloon is deflated and the apparatus, guide wire, and introducer are removed.

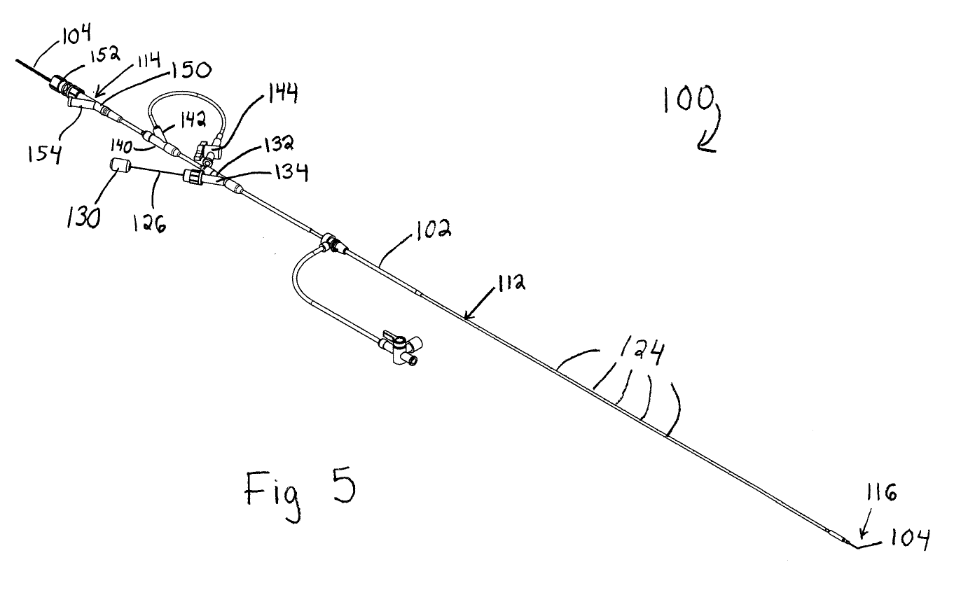

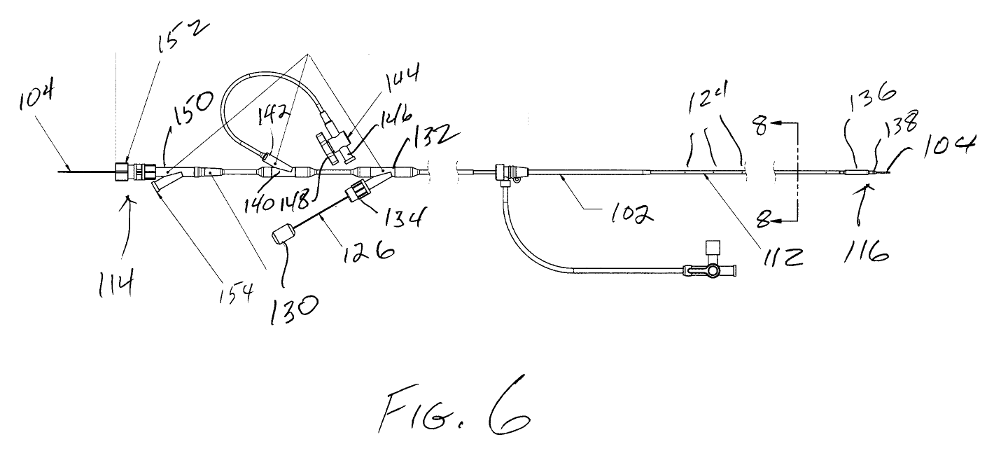

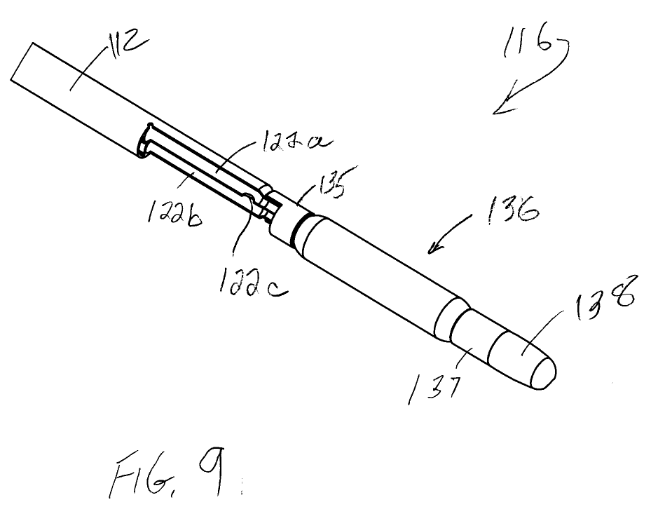

Turning now to FIGS. 5-8 a second embodiment of an apparatus according to the invention is shown. The apparatus 100 is shown together with an introducer 102 and a guide wire 104. The apparatus generally includes an elongate member 112 having a proximal end 114 and a distal end 116. As seen best in FIG. 8, the elongate member 112 has four lumens: a guide wire lumen 118, an inflation lumen 120, and a pair of infusion lumens 122a, 122b. The distal ends of the infusion lumens are coupled to each other such that infusion fluid flows distally through lumen 122b then returns proximally through lumen 122a to exit through spaced apart infusion holes 124. FIG. 9 illustrates the fluid coupling 122c which fluidly couples lumens 122a and 122b

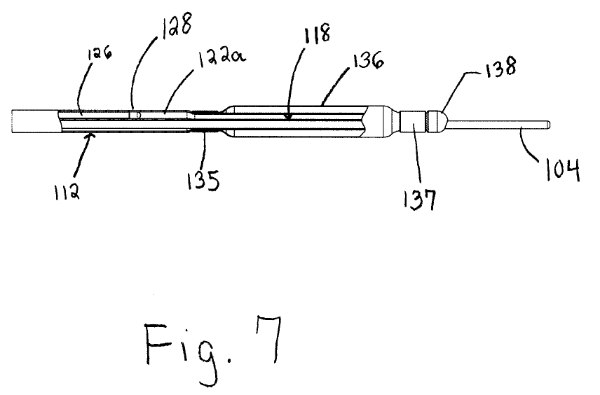

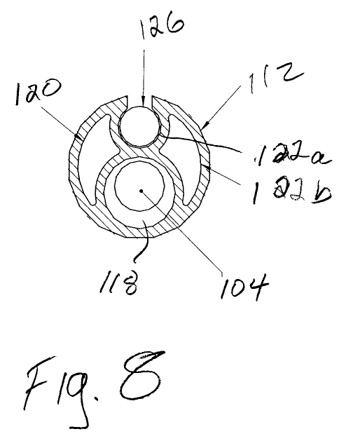

As seen best in FIGS. 7 and 8, a sliding wire 126 is disposed in the lumen 122a. The wire has a seal 128 at its distal end and a handle 130 (FIGS. 5 and 6) at its proximal end which exits the proximal end of the lumen 122a via the side port 134 of a first Y-connector 132. Those skilled in the art will appreciate that pulling on the handle 130 will move the wire and hence the seal 128 on the end of the wire 126 proximally and thereby allow infusion fluid to reach more infusion holes.

The distal end 116 of the elongate member includes an annular occlusion balloon 136 and an atraumatic tip 138. The occlusion balloon is coupled to the distal end of the inflation lumen 120 by two crimp bands 135, 137. The proximal end of the inflation lumen 120 is coupled to the side port 142 of a second Y-connector 140 which is coupled to an inflation port which has a luer 146 and a valve 148.

The guide wire lumen 118 extends from the main port 152 of a third Y-connector 150 through the annular occlusion balloon 136 and out the atraumatic tip 138. The proximal end of the infusion lumen 122b is coupled to the side port 154 of the third Y-connector 150.

From the foregoing, those skilled in the art will appreciate that the methods of using the apparatus 100 include the following steps. The blood vessel is measured and the infusion length is determined. The wire 126 is moved relative to the infusion lumen 122a to unblock a number of infusion holes corresponding to the infusion length. Though not shown in the illustration of the second embodiment, indicia could be provided on the wire 126 as were provided on the tube 22 of the first embodiment. The occlusion balloon 136 is tested and the infusion lumens 122a, 122b are primed. The sheath introducer 102 and guide wire 104 are inserted into the blood vessel to be treated. Then, the guide wire is threaded through the tip 138 into the guide wire lumen 118 and out through the port 152. The apparatus 100 is moved over the guide wire through the introducer and into the blood vessel. The distal end of the device may be radiopaque to aid in locating it with fluoroscopy. Alternatively, it can be located by measurement or by ultrasound. The guide wire is then removed from the port 152. The occlusion balloon 136 is then inflated and a therapeutic fluid is infused through the infusion port 154 such that the fluid flows distally through lumen 122b then returns proximally through lumen 122a to exit through spaced apart infusion holes 124. The balloon is deflated and the apparatus and introducer are removed.

There have been described and illustrated herein several embodiments of methods and apparatus for infusing the interior of a blood vessel. While particular embodiments of the invention have been described, it is not intended that the invention be limited thereto, as it is intended that the invention be as broad in scope as the art will allow and that the specification be read likewise. For example, while the occlusion balloon has been illustrated as coupled to the distal end of the inflation lumen by two crimp bands, other coupling means could be used. Such coupling means include glue/adhesive, sonic welding, RF welding, etc. It will therefore be appreciated by those skilled in the art that yet other modifications could be made to the provided invention without deviating from its spirit and scope as claimed.

1-20. (canceled)

21. An apparatus for treating blood vessels, comprising:
an elongate body having a proximal end, a distal end, and an infusion lumen extending there-through;
a plurality of spaced apart infusion holes along a length of the infusion lumen;
a non-perforate cylinder having an open proximal end and an open distal end and which is movable inside said infusion lumen from a first position in which access to some but not all of the infusion holes are blocked by said non-perforate cylinder to a second position where access to fewer of the infusion holes are blocked by said non-perforate cylinder, said non-perforate cylinder having a sealing structure at its distal end;
an infusion port coupled to the proximal end of said non-perforate cylinder, such that infusion fluid injected into said infusion port flows through said non-perforate cylinder, into a portion of said infusion lumen distal of said distal end of said non-perforate cylinder;
said elongate body further having a guide wire lumen extending therethrough, said infusion lumen and said guide wire lumen both having circular cross-sections;
an occlusion balloon coupled to said distal end of said elongate body and an inflation lumen extending through said elongate body and in fluid communication with said occlusion balloon, said inflation lumen having a non-circular cross-section;
said elongate body having an additional lumen extending therethrough, said additional lumen having substantially the same non-circular cross-section as said inflation lumen.

22. An apparatus according to claim 21, wherein:
each of said lumens having non-circular cross-section has two interior convex walls and one interior concave wall.

23. An apparatus according to claim 21, wherein:
a cumulative number of infusion holes are blocked when said non-perforate cylinder is moved distally and a cumulative number of infusion holes are unblocked when said non-perforate cylinder is moved proximally.

24. An apparatus according to claim 23, wherein:
said cumulative number of infusion holes are consecutive infusion holes.

25. An apparatus according to claim 24, wherein:
said non-perforate cylinder is movable inside said infusion lumen to a third position in which none of the infusion holes are blocked.

26. An apparatus according to claim 25, wherein:
said non-perforate cylinder has indicia which indicate the number of unblocked infusion holes.

27. An apparatus according to claim 21, wherein:
said infusion lumen is translucent or transparent along at least a part of its length such that the location of the distal end of the non-perforate cylinder may be ascertained.

28. (canceled)

29. An apparatus for treating blood vessels, comprising:
an elongate body having a proximal end, a distal end, and an infusion lumen extending there-through;
a plurality of spaced apart infusion holes along a length of the infusion lumen;
a non-perforate cylinder having an open proximal end and an open distal end and which is movable inside said infusion lumen from a first position in which access to some but not all of the infusion holes are blocked by said non-perforate cylinder to a second position where access to fewer of the infusion holes are blocked by said non-perforate cylinder; and
an infusion port coupled to the proximal end of said non-perforate cylinder, such that infusion fluid injected into said infusion port flows through said non-perforate cylinder, into a portion of said infusion lumen distal of said distal end of said non-perforate cylinder, wherein
said non-perforate cylinder has an annular sealing structure at its distal end.

30. An apparatus according to claim 29, wherein:
said sealing structure is a flare.

31. An apparatus according to claim 29, wherein:
said sealing structure is a balloon.

32. An apparatus according to claim 29, wherein:
said sealing structure is an annular ring made of a compliant material.
